(19) United States
(12) Patent Application Publication
(10) Pub. No.: US 2013/0149249 A1
(43) Pub. Date: Jun. 13, 2013

(54) IMAGING TUBERCULOSIS WITH PYRAZINAMIDE CONTRAST AGENTS

(71) Applicants: Vijaya Raj Kuniyil Kulangara, Bangalore (IN); Ravi Hegde, Bangalore (IN)

(72) Inventors: Vijaya Raj Kuniyil Kulangara, Bangalore (IN); Ravi Hegde, Bangalore (IN)

(73) Assignee: GE HEALTHCARE LIMITED, BUCKINGHAMSHIRE (GB)

(21) Appl. No.: 13/817,826

(22) PCT Filed: Aug. 22, 2011
(86) PCT No.: PCT/EP2011/064382

§ 371 Date: Feb. 20, 2013

Related U.S. Application Data
(60) Provisional application No. 61/391,694, filed on Oct. 11, 2010.

(30) Foreign Application Priority Data
Aug. 20, 2010 (IN) 1975/DEL/2010
Oct. 11, 2010 (GB) 1017088.4

(51) Int. Cl. C07F 13/00; A61K 51/04; C07D 241/24

(52) U.S. Cl. CPC C07F 13/005; C07D 241/24; A61K 51/0478
USPC 424/9.1; 544/225; 534/10; 534/14; 534/15; 544/406

(57) ABSTRACT

The present invention provides novel in vivo imaging agents useful for detecting the presence of mycobacteria using in vivo imaging methods. Also provided by the present invention is a precursor compound useful in the synthesis of the in vivo imaging agents of the invention, and a method to obtain the in vivo imaging agent of the invention using said precursor compound. Methods of in vivo imaging and diagnosis in which the in vivo imaging agent of the invention finds use are also provided.

### TECHNICAL FIELD OF THE INVENTION

The present invention relates to in vivo imaging and more particularly to in vivo imaging to detect the presence of tuberculosis. A novel in vivo imaging agent is provided by the present invention which has properties that make it advantageous compared with similar known in vivo imaging agents.

### DESCRIPTION OF RELATED ART

Pulmonary tuberculosis (TB) is an airborne infection caused by Mycobacterium tuberculosis (MTB) that causes high mortality and morbidity, to particularly in developing countries (Dye et al JAMA 1999; 282(7): 677-686). A recent factsheet produced by the World Health Organisation reported that the number of new cases of TB continues to increase each year in South-East Asia, the Eastern Mediterranean, and in Africa (http://www.who.int/mediacentre/factsheets/fs104/en/print.htm). The emergence of drug-resistant strains of MTB has resulted in efforts to identify new agents to treat an otherwise incurable disease.

Accurate and prompt diagnosis is important in order to control the infection and also to ensure the appropriate therapy for infected patients. Currently, a definitive diagnosis of TB requires culture of MTB from a sample taken from a patient. Patients with clear signs and symptoms of pulmonary disease with a sputum smear-positive result present no problems to diagnose. However, there can be difficulty culturing the slow-growing MTB organism in the laboratory. Furthermore the emergence of HIV has resulted in a decreased likelihood of sputum smear positivity and an increase in non-respiratory disease, such that ease of diagnosis is more difficult in these cases (see reviews by Jeong & Lee Am J Roent 2008; 191: 834-844; Davies & Pai Int J Tuberc Lung Dis 2008; 12(11): 1226-1234; and, Lange & Mori Respirology 2010; 15: 220-240).

In vivo imaging methods can be useful in the diagnosis of TB. Chest x-ray is a widely-used in vivo imaging method for screening, diagnosis and treatment monitoring in patients with known or suspected TB. Chest computed tomography (CT) is more sensitive than conventional x-ray and may be applied to identify early parenchymal lesions or mediastinal lymph node enlargements and to determine disease activity in tuberculosis (Lee & Im AJR 1995; 164(6): 1361-1367). Nuclear imaging methods have also been reported for diagnosis and treatment monitoring of TB. The positron-emission tomography (PET) tracer ¹⁸F-fluorodeoxyglucose ([¹⁸F]FDG) has been proposed as useful in the diagnosis of disease activity and therapy monitoring in patients with TB (Demura et al Eur J Nuc Med Mol Imag 2009; 36: 632-639).

Pyrazinamide (PZA) is a well-known and important first-line drug used in the treatment of TB. The mechanism of action of PZA is poorly understood, although experimental evidence suggests that PZA diffuses into MTB and is converted into pyrazinoic acid by pyrazinamidase causing pH imbalance in the bacteria followed by disruption of the bacterial cell wall (Zhang & Mitchison 2003 Int J Tuberc Lung Dis; 7(1): 6-21). There are some known teachings relating to labelled versions of PZA.

US 2008/0107598 teaches a metal chelate-targeting ligand conjugate wherein the targeting ligand can be selected from a broad variety of different pharmaceutical agents, including antimicrobial agents. Amongst a wide selection of other antimicrobial agents, PZA is disclosed as a suitable targeting ligand. However, there is no specific teaching in US 2008/0107598 as to how any specific metal chelate-PZA conjugate may be obtained.

Chohan et al (Metal-based Drugs 1998; 5(6): 347-354) teach a PZA derivative complexed with a metal selected from cobalt(II), copper(II), nickel(II) and zinc(II). The PZA derivative is disclosed as having the following structure:

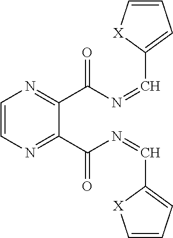

wherein X can be O, S or NH. The metal complexes are disclosed as being biologically active against one or more bacterial species, with the metal complexes being more active than the uncomplexed ligands. The ligands were not tested against MTB, and there is no disclosure in Chohan et al relating to diagnosis or in vivo imaging of TB.

Liu et al (J Med Chem 2010; 53: 2882-2891) disclose ¹¹C-labelled tuberculosis therapeutics and their use in investigating the biodistribution of these therapeutics using positron-emission tomography (PET) imaging. ¹¹C-labelled to PZA is disclosed (wherein the asterisk denotes the position of the ¹¹C label):

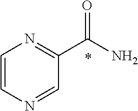

The half life of ¹¹C is relatively short (20.4 minutes) and its production is based on the nuclear reaction ¹⁴N(p,α)¹¹C on a nitrogen target that requires use of a cyclotron. The production of ¹¹C-labelled PET tracers therefore requires proximity to a cyclotron facility, which limits the geographical availability of these tracers. This is particularly true in some countries having a high incidence of TB such as India, China and African countries, where much of the population lives in remote locations. In any case, a cyclotron facility is expensive to set up and therefore not widely available. For these reasons, ¹¹C PET tracers are not ideal in the context of diagnosing TB.

KR 2007/0092536 discloses radiolabelled PZA for imaging tuberculosis, teaching that the radiolabel may be selected from ^{99m}Tc, ¹¹¹In, ¹³¹I, and ¹²³I. However, a specific structure is provided only in respect of radioiodinated PZA:

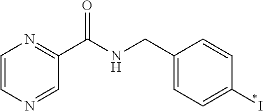 [SMILES: O=C(NCc1ccc(I)cc1)c1cnccn1]

¹²³I has a longer half-life than ¹¹C of 13.22 hours, but like ¹¹C requires a cyclotron for its production and therefore needs to be transported from a cyclotron facility to its intended site of use. This can be logistically difficult in the case of remote locations and as for this reason ¹²³I is not an ideal tracer for an in vivo TB imaging agent.

U.S. Pat. No. 5,955,053 teaches chelators which comprise a PZA moiety, e.g. L-CEPZ, to which is of the following structure:

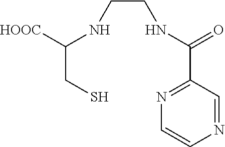 [SMILES: O=C(NCCNC(CS)C(=O)O)c1cnccn1]

When a metal ion suitable for in vivo imaging such as ^{99m}Tc is coordinated by the above structure, one of the nitrogens of the PZA moiety is involved in the coordination, as is set out in the general formula provided in claim 1 of U.S. Pat. No. 5,955,053. As such, the desired biological activity of the PZA moiety towards MTB is likely to be negatively impacted. Indeed there is no mention in U.S. Pat. No. 5,955,053 that these compounds are of use for use in the diagnosis of TB.

There is therefore scope for improved in vivo imaging agents useful in the diagnosis of TB.

### SUMMARY OF THE INVENTION

The present invention provides novel in vivo imaging agents useful for detecting the presence of mycobacteria using in vivo imaging methods. The in vivo imaging agents of the invention are radiolabelled derivatives of unlabelled small molecules that are known to have good properties as treatment agents for mycobacterial infections. The in vivo imaging agents of the present invention have similar or improved biological properties to the related unlabelled compounds. Also provided by the present invention is a precursor compound useful in the synthesis of the in vivo imaging agents of the invention, and a method to obtain the in vivo imaging agent of the invention using said precursor compound. Methods of in vivo imaging and diagnosis in which the in vivo imaging agent of the invention finds use are also provided.

### DETAILED DESCRIPTION OF THE INVENTION

### In Vivo Imaging Agent

In one aspect, the present invention provides an in vivo imaging agent of Formula I:

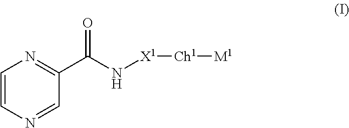

wherein:
X¹ represents a direct bond or a linker -(L)ₙ- wherein each L is independently —C(═O)—, —CR′₂—, —CR′═CR′—, —C≡C—, —CR′₂CO₂—, —CO₂CR′₂—, —NR′—, —NR′C(═O)—, —C(═O)NR′—, —NR′(C═O)NR′—, —NR′(C═S)NR′—, —SO₂NR′—, —NR′SO₂—, —CR′₂—O—CR′₂—, —CR′₂—S—CR′₂—, —CR′₂—NR′—CR′₂—, wherein each R′ group is independently H or C₁₋₆alkyl;
Ch¹-M¹ is a metal ion complex wherein Ch¹ is a chelating agent and M¹ is a metal ion suitable for in vivo imaging.

An “in vivo imaging agent” in the context of the present invention is a labelled compound that facilitates the generation of an image in an in vivo imaging procedure. The term “in vivo imaging” as used herein refers to those techniques that noninvasively produce images of all or part of the internal aspect of a subject.

The term “linker” as used herein refers to a bivalent chain of between 10 and 100 atoms, preferably between 10 and 50 atoms. Specifically excluded are linkers wherein 2 or more carbonyl groups are linked together, or wherein 2 or more heteroatoms are linked together. The skilled person would understand that these are either not chemically feasible, or are too reactive or unstable to be suitable for use in the field of the present invention. Where X¹ of Formula I represents the linker -(L)ₙ-, preferred L groups are selected from: —C(═O)—; —CH₂—; —NH—; —NHC(═O)—; —C(═O)NH—; and, —CH₂—O—CH₂—. A particularly preferred linker group to is of the formula —(CH₂)ₘ— wherein m is 1-6, preferably 2-4.

The term “metal complex” is taken to mean a coordination complex wherein a metal ion is bonded to a surrounding array of molecules or anions, which in the present invention are comprised in the chelating agent Ch¹. It is strongly preferred that the metal complex of the present invention is “resistant to transchelation”, i.e. does not readily undergo ligand exchange with other potentially competing ligands for the metal coordination sites. Potentially competing ligands include the in vivo imaging agent itself plus other excipients in the preparation in vitro (e.g. radioprotectants or antimicrobial preservatives used in the preparation), or endogenous compounds in vivo (e.g. glutathione, transferrin or plasma proteins).

A “chelating agent” is an organic compound capable of forming coordinate bonds with a metal ion through two or more donor atoms. In a typical chelating agent suitable for the present invention 2-6, and preferably 2-4, metal donor atoms are arranged such that 5- or 6-membered chelate rings result (by having a non-coordinating backbone of either carbon atoms or non-coordinating heteroatoms linking the metal donor atoms). Examples of donor atom types which bind well to metal ions as part of chelating agents are: amines, thiols, amides, oximes, and phosphines. Other arrangements are also envisaged, such as by means of ^{99m}Tc(CO)₃ radiochemistry when the metal suitable for in vivo imaging is ^{99m}Tc.

Examples of suitable chelating agents for technetium which form metal complexes resistant to transchelation include, but are not limited to:
(a) diaminedioximes;
(b) N₃S ligands having a thioltriamide donor set;
(c) N₂S₂ ligands having a diaminedithiol donor set;
(d)N₄ ligands which are open chain or macrocyclic ligands having a tetramine, amidetriamine or diamidediamine donor set; or,
(e) N₂O₂ ligands having a diaminediphenol donor set.

The above described chelating agents are particularly suitable wherein the metal ion suitable for in vivo imaging is technetium e.g. ^{94m}Tc or ^{99m}Tc, and are described more fully by Jurisson et al (Chem Rev 1999; 99: 2205-2218). These chelating to agents are also useful for other metals, such as copper (⁶⁴Cu or ⁶⁷Cu), vanadium (e.g. ⁴⁸V), iron (eg. ⁵²Fe), or cobalt (e.g. ⁵⁵Co). Other suitable chelating agents are described in Sandoz WO 91/01144, which are particularly suitable for indium, yttrium and gadolinium. Examples of such suitable chelating agents include 1,4,7,10-tetraazacyclododecane-1,4,7,10-tetraacetic acid (DOTA) and diethylenetriaminepentaacetic acid (DTPA). Non-ionic (i.e. neutral) metal complexes of gadolinium are known and are described in U.S. Pat. No. 4,885,363.

Furthermore as highlighted above, where the radioactive metal ion is ^{99m}Tc, the radioactive metal complex may also be formed by means of ^{99m}Tc(CO)₃ radiochemistry, as described more fully by Schibli in Chapter 2.2 of “Technetium-99m Pharmaceuticals: Preparation and Quality Control in Nuclear Medicine (2007 Springer; Zolle, Ed.). A benefit of this chemistry is that it can further reduce the likelihood of non-specific binding of the ^{99m}Tc to the pyrazinamide active moiety. The radioactive metal complex using this chemistry would therefore take the form of a tridentate chelate linked to ^{99m}Tc(CO)₃.

It is envisaged that the role of the linker group is to distance the relatively bulky metal complex, which results upon metal coordination, from the active site of the pyrazinamide so that e.g. substrate binding is not impaired. This can be achieved by a combination of flexibility (e.g. simple alkyl chains), so that the bulky group has the freedom to position itself away from the active site and/or rigidity such as a cycloalkyl or aryl spacer which orientates the metal complex away from the active site. The nature of the linker group can also be used to modify the biodistribution of the resulting technetium complex of the conjugate. Thus, e.g. the introduction of ether groups in the linker will help to minimise plasma protein binding, or the use of polymeric linker groups such as polyalkyleneglycol, especially polyethyleneglycol (PEG) can help to prolong the lifetime of the in vivo imaging agent in the blood in vivo. It is strongly preferred that the pyrazinamide is bound to the chelator in such a way that the linkage does not undergo facile metabolism in blood. That is because such metabolism would result in the imaging metal complex being cleaved off before the in vivo imaging agent of the invention reaches the desired in vivo target site.

A “metal ion suitable for in vivo imaging” means a metal ion that may be detected externally by an in vivo imaging technique following administration to a subject. Preferably, said metal ion suitable for in vivo imaging of Formula I is either a radioactive metal ion or a paramagnetic metal ion. When the metal ion is a radioactive metal ion it may be a gamma-emitting radioactive metal ion or a positron-emitting radioactive metal ion. A preferred gamma-emitting radioactive metal ion is selected from ^{99m}Tc, ¹¹¹In, ^{113m}In, and ⁶⁷Ga, with ^{99m}Tc being most preferred. A preferred positron-emitting radioactive metal ion is selected from ⁶⁴Cu, ⁴⁸V, ⁵²Fe, ⁵⁵Co, ^{94m}Tc and ⁶⁸Ga. When the metal ion is a paramagnetic metal ion it is preferably is selected from Gd(III), Mn(II), Cu(II), Cr(III), Fe(III), Co(II), Er(II), Ni(II), Eu(III) and Dy(III), with Gd(III) being most preferred. A most preferred metal ion suitable for in vivo imaging of the present invention is a gamma-emitting radioactive metal ion, an in particular ^{99m}Tc.

A preliminary in vitro assessment was carried out on a rhenium derivative of an imaging agent of the invention (see Example 5). The data obtained suggests that the rhenium complex is at least as active as PZA, and probably has more favourable activity than PZA.

### Pharmaceutical Composition

Preferably, the in vivo imaging agent of the invention is provided as a pharmaceutical composition together with a pharmaceutically acceptable carrier. The “pharmaceutically acceptable carrier” is a fluid, especially a liquid, in which the in vivo imaging agent is suspended or dissolved, such that the pharmaceutical composition is physiologically tolerable, i.e. can be administered to the mammalian body without toxicity or undue discomfort. The pharmaceutically acceptable carrier is suitably an injectable carrier liquid such as sterile, pyrogen-free water for injection; an aqueous solution such as to saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The pharmaceutically acceptable carrier may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

The pharmaceutical composition of the invention is suitably supplied in a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. Where the pharmaceutical composition is a radiopharmaceutical composition (i.e. when the metal ion is a gamma- or a positron-emitter), the pre-filled syringe may optionally be provided with a syringe shield to protect the operator from radioactive dose. Suitable such radiopharmaceutical syringe shields are known in the art and preferably comprise either lead or tungsten.

The pharmaceutical composition of the present invention may be prepared from a kit, as is described below in an additional aspect of the invention. Alternatively, it may be prepared under aseptic manufacture conditions to give to the desired sterile product. The pharmaceutical composition may also be prepared under non-sterile conditions, followed by terminal sterilisation using e.g. gamma-irradiation, autoclaving, dry heat or chemical treatment (e.g. with ethylene oxide). Preferably, the pharmaceutical composition of the present invention is prepared from a kit.

### Precursor Compound

In another aspect, the present invention provides a precursor compound of Formula II:

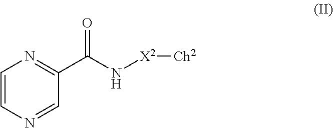

wherein X² is as defined herein for X¹, and Ch² is as defined herein for Ch¹.

The suitable and preferred embodiments of X¹ and Ch¹ are equally applicable to X² and Ch².

A “precursor compound” comprises a derivative of the in vivo imaging agent of Formula I wherein the metal ion is not complexed by the chelating agent. Such precursor compounds are typically designed so that chemical reaction with a convenient chemical form of the metal ion occurs site-specifically; can be conducted in the minimum number of steps (ideally a single step); and without the need for significant purification (ideally no further purification), to give the desired in vivo imaging agent. Such precursor compounds are synthetic and can conveniently be obtained in good chemical purity. In order to facilitate site-specific reaction, the precursor compound of the invention may optionally comprise a suitable protecting group.

By the term “protecting group” is meant a group which inhibits or suppresses undesirable chemical reactions, but which is designed to be sufficiently reactive that it may be cleaved from the functional group in question to obtain the desired product under mild enough conditions that do not modify the rest of to the molecule. Protecting groups are well known to those skilled in the art and are described in ‘Protective Groups in Organic Synthesis’, Theorodora W.

Greene and Peter G. M. Wuts, (Fourth Edition, John Wiley & Sons, 2007).

Precursor compounds of the present invention may be obtained in a straightforward manner starting by coupling a commercially-available pyrazine derivative with a suitable derivative of the desired chelate e.g. as illustrated below in Scheme 1:

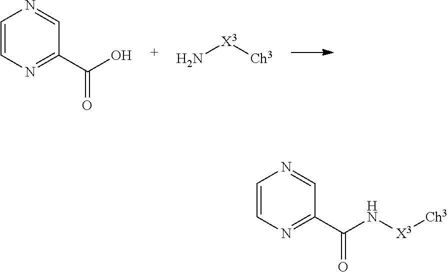

wherein X³ and Ch³ are as suitably and preferably defined herein for X¹ and Ch¹, respectively. Reaction conditions suitable for the coupling reaction are well-known to the person skilled in the field of organic chemistry (see March's Advanced Organic Chemistry 6^{th} Edition; Wiley: Smith & March, Eds.).

The precursor compound of the invention is ideally provided in sterile, apyrogenic form. The precursor compound can accordingly be used for the preparation of the above-described pharmaceutical composition of the invention, and for inclusion as a component in a kit for the preparation of such a pharmaceutical composition, as described in greater detail below.

### Method of Preparation

In a further aspect, the present invention provides a method for the preparation of the in vivo imaging agent as defined herein wherein said method comprises:
(i) providing a precursor compound of Formula II as defined herein;
(ii) reacting said precursor compound with a source of a metal ion suitable for in vivo imaging wherein said metal ion is as defined herein.

The step of “reacting” the precursor compound with the source of a metal ion suitable for in vivo imaging involves bringing the two reactants together under reaction conditions suitable for formation of the desired in vivo imaging agent to in as high a radiochemical yield (RCY) as possible. Synthetic routes for obtaining particular in vivo imaging agents of the present invention are presented in the experimental section below.

The term “source of a metal ion” refers to the metal ion in a chemical form that will react in a single step with the precursor compound of the invention to form the metal chelate -Ch¹-M¹ of Formula I. For example, when the metal ion is technetium, the usual source of technetium for labelling is pertechnetate, i.e. TcO₄—, which is technetium in the Tc(VII) oxidation state. Pertechnetate itself does not readily form complexes, hence the preparation of technetium complexes usually requires the addition of a suitable reducing agent such as stannous ion to facilitate complexation by reducing the oxidation state of the technetium to the lower oxidation states, usually Tc(I) to Tc(V). The solvent may be organic or aqueous, or mixtures thereof. When the solvent comprises an organic solvent, the organic solvent is preferably a biocompatible solvent, such as ethanol or dimethylsulfoxide (DMSO). Preferably the solvent is aqueous, and is most preferably isotonic saline. Where it is desired to label with Gd(III), Gd₂O₃ can be reacted with the precursor compound of the invention. The person skilled in the art of in vivo imaging agents will be familiar with other sources of metal ion that are suitable for application in the present invention. For more detail, the reader is referred to the “Handbook of Radiopharmaceuticals” (2003; Wiley: Welch and Redvanly, Eds), and to “Contrast Agents: Magnetic Resonance Imaging” (2002; Springer-Verlag: Krause, Ed).

The suitable and preferred embodiments of the in vivo imaging agent of the invention and precursor compound of the invention as applied to this aspect of the invention are as defined above.

### Kit

In a yet further aspect, the present invention provides a kit for carrying out the above-described method for the preparation of the in vivo imaging agent of the invention, and preferably for preparing the pharmaceutical composition of the invention, wherein said kit comprises a vial containing the precursor compound of the invention as defined herein.

The precursor compound of the invention is preferably provided in the kit of the invention in sterile non-pyrogenic form, so that reaction with a sterile source of a suitable metal ion gives the desired pharmaceutical with the minimum number of manipulations. Such considerations are particularly important in the case of radiopharmaceuticals, in particular for radiopharmaceuticals where the radioisotope has a relatively short half-life, for ease of handling and hence reduced radiation dose for the radiopharmacist. Hence, the reaction medium for reconstitution of such kits is preferably a “pharmaceutically acceptable carrier” as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

Preferred aspects of the precursor compound of the invention when employed in the kit are as herein described. The precursor compound for use in the kit may be employed under aseptic manufacture conditions to give the desired sterile, non-pyrogenic material. The precursor compound may also be employed under non-sterile conditions, followed by terminal sterilisation using e.g. gamma-irradiation, autoclaving, dry heat or chemical treatment (e.g. with ethylene oxide). Preferably, the precursor compound is employed in sterile, non-pyrogenic form. Most preferably the sterile, non-pyrogenic precursor compound is employed in the sealed container as described above.

For ^{99m}Tc, the kit is preferably lyophilised and is designed to be reconstituted with sterile ^{99m}Tc-pertechnetate (TcO⁴⁻) from a ^{99m}Tc radioisotope generator to give a to solution suitable for human administration without further manipulation. Suitable kits comprise a container (e.g. a septum-sealed vial) containing the uncomplexed chelating agent, together with a pharmaceutically acceptable reducing agent such as sodium dithionite, sodium bisulphite, ascorbic acid, formamidine sulphinic acid, stannous ion, Fe(II) or Cu(I); together with at least one salt of a weak organic acid with a pharmaceutically acceptable cation. By the term “pharmaceutically acceptable cation” is meant a positively charged counterion which forms a salt with an ionised, negatively charged group, where said positively charged counterion is also non-toxic and hence suitable for administration to the mammalian body, especially the human body. Examples of suitable pharmaceutically acceptable cations include: the alkali metals sodium or potassium; the alkaline earth metals calcium and magnesium; and the ammonium ion. Preferred pharmaceutically acceptable cations are sodium and potassium, most preferably sodium.

The kits for preparation of ^{99m}Tc imaging agents may optionally further comprise a second, weak organic acid or salt thereof with a biocompatible cation, which functions as a transchelator. The transchelator is a compound which reacts rapidly to form a weak complex with technetium, then is displaced by the chelator of the kit. This minimises the risk of formation of reduced hydrolysed technetium (RHT) due to rapid reduction of pertechnetate competing with technetium complexation. Suitable such transchelators are the weak organic acids and salts thereof described above, preferably tartrates, gluconates, glucoheptonates, benzoates, or phosphonates, preferably phosphonates, most especially diphosphonates. A preferred such transchelator is MDP, ie. methylenediphosphonic acid, or a salt thereof with a biocompatible cation.

Also in relation to ^{99m}Tc kits, the kit may optionally contain a non-radioactive metal complex of the chelator which, upon addition of the technetium, undergoes transmetallation (i.e. ligand exchange) giving the desired product. Suitable such complexes for transmetallation are copper or zinc complexes.

The pharmaceutically acceptable reducing agent used in the ^{99m}Tc imaging agent kit is preferably a stannous salt such as stannous chloride, stannous fluoride or to stannous tartrate, and may be in either anhydrous or hydrated form. The stannous salt is preferably stannous chloride or stannous fluoride.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a pharmaceutically acceptable cation. The “pharmaceutically acceptable cation” and preferred embodiments thereof are as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution. The antimicrobial preservative may, however, also optionally be used to inhibit the growth of potentially harmful micro-organisms in one or more components of the kit prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS (i.e. tris(hydroxymethyl)aminomethane), and pharmaceutically acceptable bases such as sodium carbonate, sodium bicarbonate to or mixtures thereof. When the precursor compound is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

### Clinical Use

The in vivo imaging agent of the invention finds use in the diagnosis and monitoring of infection caused by Mycobacteria tuberculosis (MTB). Accordingly, the present invention provides a method to determine the location and/or amount of MTB present in a subject, wherein said method comprises:
(a) administering the in vivo imaging agent as defined herein to said subject in an amount suitable for in vivo imaging;
(b) allowing the administered in vivo imaging agent to bind to any MTB present in said subject;
(c) detecting by a suitable in vivo imaging procedure signals emitted by said metal ion suitable for in vivo imaging comprised in said in vivo imaging agent;
(d) generating an image representative of the location and/or amount of said signals; and,
(e) attributing the location and/or amount of said signals to the location and/or amount of MTB present in said subject.

“Administering” the in vivo imaging agent is preferably carried out parenterally, and most preferably intravenously. The intravenous route represents the most efficient way to deliver the in vivo imaging agent throughout the body of the subject, and also does not represent a substantial physical intervention on the body of the subject. By the term “substantial” is meant an intervention which to requires professional medical expertise to be carried out, or which entails a substantial health risk even when carried out with the required professional care and expertise. The in vivo imaging agent of the invention is preferably administered as the pharmaceutical composition of the invention, as defined herein. The in vivo imaging method of the invention can also be understood as comprising the above-defined steps (b)-(e) carried out on a subject to whom the in vivo imaging agent of the invention has been pre-administered.

Following the administering step and preceding the detecting step, the in vivo imaging agent is allowed to bind to any MTB present within said subject. For example, when the subject is an intact mammal, the in vivo imaging agent will dynamically move through the mammal's body, coming into contact with various tissues therein. Once the in vivo imaging agent comes into contact with MTB, the two entities bind such that clearance of the in vivo imaging agent from tissue in which MTB is present takes longer than from tissue without any, or less, MTB present. A certain point in time will be reached when detection of in vivo imaging agent specifically bound to MTB is enabled as a result of the ratio between in vivo imaging agent bound to tissue with MTB versus that bound in tissue without any MTB. This is the optimal time for the detecting step to be carried out.

The “detecting” step of the method of the invention involves detection of signals emitted by the radioisotope by means of a detector sensitive to said signals. This detection step can also be understood as the acquisition of signal data. For gamma-emitting metal ions, single-photon emission tomography (SPECT) is used; for positron-emitting metal ions, positron-emission tomography (PET) is used; and, for paramagnetic metal ions, magnetic resonance imaging (MRI) is used.

The “generating” step of the method of the invention is carried out by a computer which applies a reconstruction algorithm to the acquired signal data to yield a dataset. This dataset is then manipulated to generate images showing the location and/or amount of signals emitted by the metal ion which is comprised in the in vivo imaging agent. The signals emitted directly correlate with the presence of MTB such that the “determining” step can be made by evaluating the generated to image.

The “subject” of the invention can be any human or animal subject. Preferably the subject of the invention is a mammal. Most preferably, said subject is an intact mammalian body in vivo. In an especially preferred embodiment, the subject of the invention is a human. The in vivo imaging method may be used in subjects known or suspected to have TB caused by MTB. The in vivo imaging method of the invention may be carried out repeatedly during the course of a treatment regimen for said subject, said regimen comprising administration of a drug to combat TB caused by MTB.

In another aspect, the present invention provides the pharmaceutical composition of the invention for use in a method to determine the location and/or amount of MTB present in a subject, wherein said method is as previously defined herein.

Furthermore, the present invention provides for use of the in vivo imaging agent of the invention in the manufacture of the pharmaceutical composition of the invention for use in a method to determine the location and/or amount of MTB present in a subject, wherein said method is as previously defined herein.

### BRIEF DESCRIPTION OF THE EXAMPLES

Example 1 describes the synthesis of a starting material for the preparation of a precursor of the invention.

Example 2 describes how a particular precursor compound of the invention was obtained.

Example 3 describes how the precursor compound from Example 1 was labelled with rhenium.

Example 4 describes how the precursor compound of Example 1 can be radiolabelled with ^{99m}Tc to obtain an in vivo imaging agent of the present invention.

Example 5 describes the in vitro assessment of a rhenium derivative of a compound of the invention.

### LIST OF ABBREVIATIONS USED IN THE EXAMPLES

DCM dichloromethane

EDTA ethylenediaminetetraacetic acid

HPLC high-performance liquid chromatography

MeOH methanol

mg milligram(s)

mL millilitre(s)

mmol millimole(s)

MS mass spectrometry

MSA methanesulfonic acid

NMR nuclear magnetic resonance

RT room temperature

THF tetrahydrofuran

### EXAMPLES

### Example 1

### N′-[2-(4-Methoxy-benzylsulfanyl)-ethyl]-N′-{2-[4-methoxy-benzylsulfanyl)-ethylamino]-ethyl}-propan-1,3-diamine (1)

### 1(a) Synthesis of 2-(4-Methoxy-benzylsulfanyl)-ethylamine

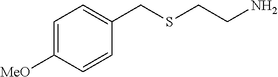 [SMILES: COc1ccc(CSCCN)cc1]

Sodium (1.50 g, 65.2 mmol) was added in small portions (about 100 mgs) under a blanket of nitrogen to vigorously stirred methanol (50 ml HPLC grade). When the effervescence had ceased 2-aminoethanethiol hydrochloride (3.6 g 31.6 mmol) was to added in one portion precipitating sodium chloride from solution. p-methoxybenzyl chloride (5.0 g, 32.0 mmol) was added in one portion and the mixture heated under reflux at 75-80° C. for 30 minutes. After cooling the solid was removed by filtration and the filter cake washed with methanol (15 ml). The organic extracts were combined and volatiles removed under reduced pressure (10 mm Hg, 40° C.) to leave a colourless oil which contained sodium chloride crystals. This residue was redissolved in DCM (25 ml), extracted with water (25 ml×3), dried (MgSO₄), filtered and solvent removed to leave a colourless oil. This material was about (95%) of desired compound and not purified any further. Yield 5.4 g (87%).

¹H-NMR (CDCl₃) δ 1.25 (2H, bs, NH₂), 2.42 (2H, t, J=7 Hz, SCH₂), 2.73 (2H, t, J=7 Hz, CH₂N), 2.73 (2H, s, SCH₂Ar), 3.74 (3H, s, OCH₃), 6.77 (2H, d, J=7 Hz, CH×2), 7.15 (2H, d, J=7 Hz, CH×2).

### 1(b) Synthesis of N-{(4-Methoxy-benzylsulfanyl)-ethyl}-2-chloroacetamide

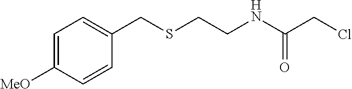 [SMILES: COc1ccc(CSCCNC(=O)CCl)cc1]

Chloroacetyl chloride (630 mg, 5.6 mmol) in dry DCM (5 ml) was added dropwise over 5 minutes with stirring to an ice bath cooled (0-5° C.) solution of 2-(4-Methoxy-benzylsulfanyl)-ethylamine (1.0 g, 5 mmol) and triethylamine (600 mg, 5.9 mmol) in dry DCM (20 ml). After addition the cooling bath was removed and stirring continued for 30 minutes. The solution was extracted with water (50 ml×2), dried (MgSO₄), filtered and solvent evaporated under reduced pressure to leave a fawn coloured solid. Yield 1.30 g (95%). This material required no further purification.

¹H-NMR (CDCl₃): δ 2.54 (2H, t, J=7 Hz, SCH₂), 3.39 (2H, t, J=7 Hz, NCH₂), to 3.66 (2H, s, SCH₂Ar), 3.99 (3H, s, OCH₃), 6.82 (2H, d, J=8 Hz, CH×2), 6.90 (1H, bs, NH), 7.21 (2H, d, J=8 Hz, CH×2)

### 1(c) Methyl 3-[(4-Methoxy-benzylsufanyl)-ethylamino]propanoate

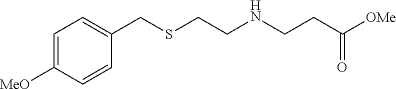 [SMILES: COC(=O)CCNCCSCc1ccc(OC)cc1]

Methyl acrylate (440 mg 5.1 mmol) in methanol (1 ml) was added in one portion to a stirred solution of 2-(4-Methoxy-benzylsulfanyl)-ethylamine (1.0 g, 5 mmol) in methanol (5 ml). The colourless solution was allowed to stir at RT for 2 hours. Volatiles were removed by rotary evaporation to leave the product as colourless viscous oil in about 95% purity. Yield 1.35 g (96%). An analytical sample was obtained by chromatography over silica eluting with DCM/MeOH 98:2. The product was isolated as a colourless viscous oil r_{f}=0.2.

¹H-NMR (CDCl₃) δ 1.67 (1H, bs, NH), 2.45 (2H, t, J=7 Hz, CH₂C═O), 2.53 (2H, t, J=7 Hz, SCH₂), 2.72 (2H, t, J=7 Hz, NCH₂), 2.81 (2H, t, J=7 Hz, NCH₂), 3.64 (2H, s, SCH₂—C—N), 3.66 (3H, s, OCH₃ ester), 3.75 (3H, s, OCH₃ methoxy), 6.80 (2H, d, J=8 Hz, CH×2), 7.20 (2H, d, J=8 Hz, CH×2).

### 1(d) 3-[2-(4-Methoxybenzylsulfanyl)-ethylamino]propanamide

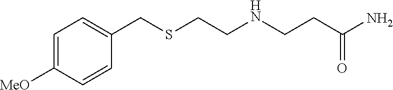 [SMILES: COc1ccc(CSCCNCCC(N)=O)cc1]

Methyl 3-[(4-Methoxy-benzylsufanyl)-ethylamino]propanoate (1.35 g 4.8 mmol), methanol (15 ml) and ammonia solution (25 ml) were stirred at RT for 16 hours.

Volatiles were removed under reduced pressure (10 mmHg, 50° C.) to leave a viscous oil which solidifies on standing. Yield 1.28 g (99%). This material is about 95% pure and can be used in the next step. An analytical sample was obtained after purification by chromatography over silica eluting with DCM/Methanol 80:20. The product had a r_{f}=0.15 and was isolated as a white solid.

¹H-NMR (CDCl₃) δ 2.54 (2H, t, J=7 Hz, CH₂C═O), 2.65 (2H, t, J=7 Hz, SCH₂—C—N), 2.91 (2H, t, J=7 Hz, NCH₂), 3.00 (2H, t, J=7 Hz, NCH₂), 3.84 (2H, s, SCHPh), 3.95 (3H, s, OCH₃), 7.00 (2H, d, J=8 Hz, CH×2), 7.48 (2H, d, J=8 Hz, CH×2)

### 1(e) 3-([2-(4-Methoxy-benzylsulfanyl)-ethyl]-{[2-(4-methoxy-benzylsulfanyl)-ethylcarbamoyl]-methyl}-amino)propionamide

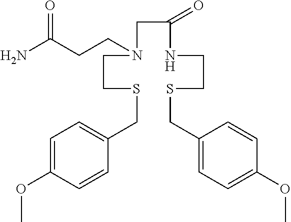 [SMILES: COc1ccc(CSCCNC(=O)CN(CCSCc2ccc(OC)cc2)CCC(N)=O)cc1]

3-[2-(4-Methoxybenzylsulfanyl)-ethylamino]propanamide (670 mgs, 2.5 mmol), N-{(4-Methoxy-benzylsulfanyl)-ethyl}-2-chloroacetamide (680 mgs, 2.5 mmol), trimethylamine (300 mgs 3 mmol) and acetonitrile (5 mls) were heated under reflux at 70° C. for 16 hours. The solvent was removed under reduced pressure to leave an orange/brown residue which was redissolved in DCM (25 ml), extracted with water (25 ml×2), dried (MgSO₄), filtered and solvent evaporated by rotary evaporation. The residue was purified over silica eluting with DCM/Methanol 95:5 (r_{f}=0.15) to give a colourless viscous oil (Yield 450 mg 36%).

¹H-NMR (CDCl₃) δ 2.27 (2H, t, J=7 Hz, CH₂CO), 2.51 (2H, t, J=7 Hz, SCH₂), 2.55 (2H, t, J=7 Hz, SCH₂), 2.62 (2H, t, J=7 Hz, NCH₂), 2.76 (2H, t, J=7 Hz, NCH₂), 3.04 (2H, s, NCH₂CO), 3.39 (2H, q, J=6 Hz, H₂CNH), 3.62 (2H, s, SCH₂Ph), 3.65 ((2H, s, SCH₂Ph), 3.76 (6H, s, OMe×2), 5.52 (1H bs, NH), 5.97 (1H, bs, NH), 6.81 (4H, bd, J=8 Hz, CH×4), 7.18 (2H, d, J=8 Hz, CH×2), 7.20 (2H, d, J=8 Hz, CH×2), 7.70 (1H, bt, J=6 Hz, HNCO)

### 1(f) N′-[2-(4-Methoxy-benzylsulfanyl)-ethyl]-N′-{2-[4-methoxy-benzylsulfanyl)-ethylamino]-ethyl}-propan-1,3-diamine (1)

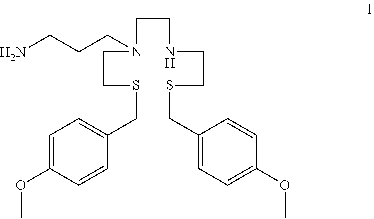 [SMILES: COc1ccc(CSCCNCCN(CCCN)CCSCc2ccc(OC)cc2)cc1]

1.0M Borane in THF (12 mls, 12 mmol) was added via a syringe under a nitrogen atmosphere to 3-([2-(4-Methoxy-benzylsulfanyl)-ethyl]-{[2-(4-methoxy-benzylsulfanyl)-ethylcarbamoyl]-methyl}-amino) propionamide (450 mgs, 0.89 mmol). The resulting colourless solution was heated under reflux at 70° C. A white gum precipitated after about 40 minutes but heating was continued for a further 16 hours. After cooling to RT water (2 mls) was added dropwise allowing for the vigorous effervescence to cease. The solvent was removed under reduced pressure to leave a waxy solid to which dilute HCl (2%, 20 mls) was added and the mixture heated under reflux at 100° C. for 3 hours. After cooling, sodium hydroxide was added until a pH 10-11 was obtained. This mixture was extracted with DCM (25 mls×3) and the fractions combined, dried (MgSO₄), filtered and solvent evaporated to leave a waxy solid. This material was analysed by ¹³C nmr and found to be a mixture of mono and di reduced products. This material was subjected to silica gel chromatography eluting with DCM/MeOH/NH₄OH 90:10:1. The product eluted with an r_{f}=00.35 and was isolated as a colourless oil. Yield 180 mg (42%).

¹H-NMR (CDCl₃) δ 1.48 (2H, quintet, J=7 Hz, —CH₂—), 2.29-2.52 (15H, SCH₂×2+4×CH₂N+NH₂+NH, m), 2.64 (2H, t, J=7 Hz, NCH₂), 2.67 (2H, t, J=7 Hz, NCH₂), 3.59 (4H, s, SCH₂Ph×2), 3.70 (6H, s, OCH₃×2), 6.76 (4H, d, J=9 Hz, CH×4), 7.15 (4H, d, J=9 Hz, CH×4).

### Example 2

### Precursor Compound 1(4-(hydrzinecarbonll)-N-(3-((2-(4-methoxylbenzylthio)ethyl)(2-(2-(4-methoxybenzylthio)ethylamino)ethyl)amino)propyl)picolinamide)

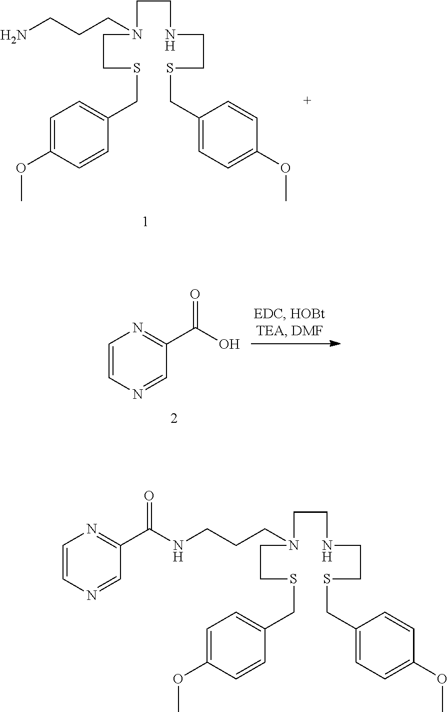 [SMILES: COc1ccc(CSCCNCCN(CCCN)CCSCc2ccc(OC)cc2)cc1 ; O=C(O)c1cnccn1 ; COc1ccc(CSCCNCCN(CCCNC(=O)c2cnccn2)CCSCc2ccc(OC)cc2)cc1]

In to a mixture of pyrazinoic acid (2 15.4 mg, 0.125 mmol), 1-ethyl-3-[3-dimethylaminopropyl]carbodiimide hydrochloride (30 mg, 0.156 mmol), hydroxybenzotriazole (2.8 mg, 0.02 mmol) and triethylamine (21 mg, 0.208 mmol) in dimethylformamide was added 1(50 mg, 0.104 mmol) slowly and allowed to stir at RT overnight. The reaction mixture was concentrated, dissolved in ethylacetate and washed with water. The organic layer was dried with sodium sulphate, concentrated and purified by combiflash chromatography. Yield, 21 mg (29%). ¹H NMR (CDCl₃): δ. 9.37 (s, 1H), 8.73-8.77 (d, 1H), 8.5 (s, 1H), 7.17-2.27 (dd, 4H), 6.77-6.87 (dd, 4H), 3.78 (s, 6H), 3.72 (s, 2H), 3.66 (s, 2H), 3.52-3.6 (m, 2H), 2.48-3.02 (m, 14H) and 1.72-1.82 (m, 2H). MS (m/z): 584.6 (M+H⁺).

### Example 3

### Re-Labelling of Precursor Compound 1

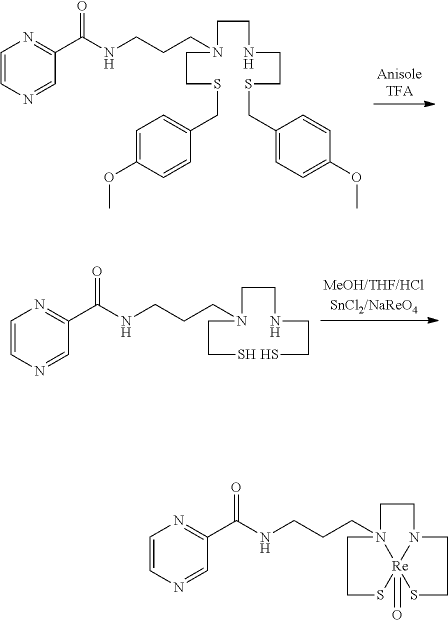

The methods described by Zhen et al (J Med Chem 1999; 42: 2805-2815) were adapted to label precursor compound 1 with rhenium. Precursor compound 1, 70 mg (0.117 mmol) was dissolved in 1 mL of trifluoroacetic acid (TFA), added one drop of anisole and 0.2 mL of MSA. The reaction mixture was stirred at 60° C. for 90 minutes under nitrogen atmosphere. Later it was concentrated under vacuum for 2 hours to remove TFA/anisole/MSA. Resulting mass was taken in 14 mL of 7:1 CH/OH:THF and heated to reflux. Tin(II) chloride (49 mg, to 0.25 mmol, in 300 uL of 0.1 M HCl) was added, followed immediately by a solution of sodium perrhenate (70 mg, 0.25 mmol, in 300 uL of distilled water). Refluxing was continued for 16 hours, after which the solution was filtered and concentrated. The product was purified by preparative HPLC to a purity of 99%. MS (m/z): 558.3 (M+H⁺).

### Example 4

### ^{99m}Tc-Labelling of Precursor Compound 1

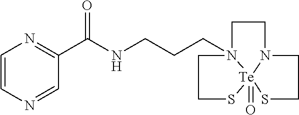

The ^{99m}Tc-labelling conditions described by Meegalla et ac (J Med Chem 1998; 41: 428-436) can be used to label precursor compound with ^{99m}Tc. In summary, precursor compound 1 (0.2-0.4 μmol) is treated with TFA/anisole/MSA as per the procedure given in Example 3 above. After the removal of the volatiles, the residue is dissolved in 100 μL of EtOH and 100 μL of HCl (1 N). HCl (500 μL, 1 N), 1 mL of Sn-glucoheptonate solution (containing 136 μg of SnCl₂ and 200 μg of Naglucoheptonate, pH 6.67), and 50 μL of EDTA solution (0.1 N) are successively added. [^{99m}Tc]Pertechnetate (100-200 μL; from 1-20 mCi) in saline solution is then added. The reaction mixture is heated for 30 min at 100° C. (or 121° C. in an autoclave for 30 min), cooled to RT, and neutralized with a saturated NaHCO₃ solution. After the complex is extracted from the aqueous reaction medium with ethyl acetate (1×3, 2×1.5 mL) and passed through a small column of Na₂SO₄, the ethyl acetate extracts are to condensed under a flow of N₂. The residue is dissolved in 200 μL of EtOH and purified by HPLC.

### Example 5

### In Vitro Assessment

A series of in vitro tests were carried out to determine the properties of known compounds alongside a rhenium derivative of an in vivo imaging agent of the present invention. These tests were the Microplate Alamar Blue Assay (MABA) (Franzblau S et al., J Clin Microbiol 1998, 36, 362-366), the Low Oxygen Recovery Assay (LORA) (Cho et al., Antimicro Agents Chemother 2007, 51, 1380-1385), and the VERO cell cytotoxicity assay to determine IC 50 (Cory A H et al., Cancer Commun 1991, 3, 207-12).

### 5(a) Microplate Alamar Blue Assay (MABA)

The initial screen was conducted against Mycobacterium tuberculosis H37Rv (ATCC 27294) in BACTEC 12B medium using the Microplate Alamar Blue Assay (MABA). Compounds were tested in ten 2-fold dilutions, typically from 100 μg/mL to 0.19 μg/mL at pH 6.8. The MIC90 is defined as the concentration effecting a reduction in fluorescence of 90% relative to controls. This value was determined from the dose-response curve using a curve-fitting program.

### 5(b) Low Oxygen Recovery Assay (LORA)

This assay was done with single concentration, typically at ≧10 uM or ug/ml vs. hypoxia-adapted M. tuberculosis H37Rv carrying luciferase gene; exposure for 10 days to test compound under anaerobic conditions; luminescent % inhibition readout.

### 5(c) Method for Determining IC₅₀

The VERO cell cytotoxicity assay was done in parallel with the TB Dose Response assay. After 72 hours exposure, viability was assessed using Promega's Cell Titer Glo Luminescent Cell Viability Assay, a homogeneous to method of determining the number of viable cells in culture based on quantitation of the ATP present. Cytotoxicity was determined from the dose-response curve as the IC₅₀ using a curve-fitting program.

Table 1 below sets out the data obtained for the parent PZA compound, the chelator obtained by the method of Example 1(f) above, PZA conjugated to said chelate obtained by the method of Example 2, and a rhenium complex of PZA conjugated to said chelate obtained by the method of Example 3.

| | MABA MIC (90) | | IC50 |
|---|---|---|---|
| Analog | (ug/ml) | LORA MIC (90) | (ug/ml) |
| Standard drug | >100 | NA | NA |
| Chelate alone | 1.49 | 0.531 | >50 |
| PZA + Chelate | 10.385 | 10.31 | 36.84 |
| Rhenium complex | >50 | >50 | >50 |

What is claimed is:

1) An in vivo imaging agent of Formula I: 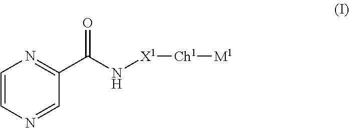
wherein:
X¹ represents a direct bond or a linker -(L)ₙ- wherein each L is independently —C(═O)—, —CR′₂—, —CR′═CR′—, —C≡C—, —CR′₂CO₂—, —CO₂CR′₂—, —NR′—, —NR′CO—, —CONR′—, —NR′(C═O)NR′—, —NR′(C═S)NR′—, —SO₂NR′—, —NR′SO₂—, —CR′₂OCR′₂—, —CR′₂SCR′₂—, —CR′₂NR′CR′₂—, wherein each R′ group is independently H or C₁₋₆ alkyl;
Ch¹-M¹ is a metal ion complex wherein Ch¹ is a chelating agent and M¹ is a metal ion suitable for in vivo imaging.

2) The in vivo imaging agent as defined in claim 1 wherein said metal ion suitable for in vivo imaging is either a radioactive metal ion or a paramagnetic metal ion.

3) The in vivo imaging agent as defined in claim 2 wherein said radioactive metal ion is a gamma-emitting radioactive metal ion selected from ^{99m}Tc, ¹¹¹In, ^{113m}In, and ⁶⁷Ga.

4) The in vivo imaging agent as defined in claim 3 wherein said radioactive metal ion is ^{99m}Tc.

5) The in vivo imaging agent as defined in claim 2 wherein said radioactive metal ion is a positron-emitting radioactive metal ion selected from ⁶⁴Cu, ⁴⁸V, ⁵²Fe, ⁵⁵Co, ^{94m}Tc and ⁶⁸Ga.

6) The in vivo imaging agent as defined in claim 2 wherein said paramagnetic metal ion is selected from Gd(III), Mn(II), Cu(II), Cr(III), Fe(III), Co(II), Er(II), Ni(II), Eu(III) and Dy(III).

7) The in vivo imaging agent as defined in claim 1 wherein Ch¹ is:
(a) diaminedioximes;
(b) N₃S ligands having a thioltriamide donor set;
(c) N₂S₂ ligands having a diaminedithiol donor set;
(d) N₄ ligands which are open chain or macrocyclic ligands having a tetramine, amidetriamine or diamidediamine donor set; or,
(e) N₂O₂ ligands having a diaminediphenol donor set.

8) A pharmaceutical composition comprising the in vivo imaging agent as defined in claim 1 together with a pharmaceutically acceptable carrier.

9) A precursor compound of Formula II: 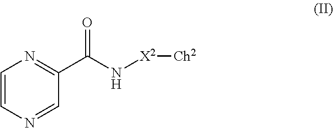
wherein X² is as defined in claim 1 for X¹, and Ch² is as defined for Ch¹ in claim 1.

10) A method for the preparation of the in vivo imaging agent as defined in claim 1 wherein said method comprises:
(i) providing a precursor compound of Formula II 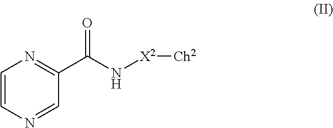
wherein X² is as defined in claim 1 for X¹, and Ch² is as defined for Ch¹ in claim 1;
(ii) reacting said precursor compound with a source of a metal ion suitable for in vivo imaging.

11) The method as defined in claim 10 wherein said source of metal ion suitable for in vivo imaging is a source of ^{99m}Tc.

12) The method as defined in claim 11 wherein said source of ^{99m}Tc is pertechnetate.

13) A kit comprising a vial containing the precursor compound as defined in claim 9.

14) A method to determine the location and/or amount of Mycobacteria tuberculosis (MTB) present in a subject, wherein said method comprises:
(a) administering the in vivo imaging agent as defined in claim 1 to said subject in an amount suitable for in vivo imaging;
(b) allowing the administered in vivo imaging agent to bind to any MTB present in said subject;
(c) detecting by a suitable in vivo imaging procedure signals emitted by said metal ion suitable for in vivo imaging comprised in said in vivo imaging agent;
(d) generating an image representative of the location and/or amount of said signals; and,
(e) attributing the location and/or amount of said signals to the location and/or amount of MTB present in said subject.

15) The method as defined in claim 14 wherein said administration step is carried out by intravenous injection.

16) The method as defined in claim 14 wherein said in vivo imaging agent is administered as a pharmaceutical composition comprising an in vivo imaging agent of Formula I 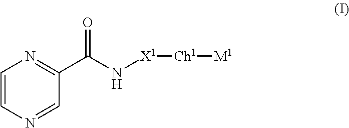
wherein:
X¹ represents a direct bond or a linker -(L)ₙ- wherein each L is independently —C(═O)—, —CR′₂—, —CR′═CR′—, —C≡C—, —CR′₂CO₂—, —CO₂CR′₂—, —NR′—, —NR′CO—, —CONR′—, —NR′(C═O)NR′—, —NR′(C═S)NR′—, —SO₂NR′—, —NR′SO₂— —CR′₂OCR′₂—, —CR′₂SCR′₂—, —CR′₂NR′CR′₂—, wherein each R′ group is independently H or C₁₋₆ alkyl;
Ch¹-M¹ is a metal ion complex wherein Ch¹ is a chelating agent and M¹ is a metal ion suitable for in vivo imaging,
together with a pharmaceutically acceptable carrier.

17) The method as defined in claim 14 which is carried out repeatedly during the course of a treatment regimen for said subject, said regimen comprising administration of a drug to combat TB caused by MTB.

18) (canceled)

19) (canceled)
